(19) United States
(12) Patent Application Publication
(10) Pub. No.: US 2024/0293317 A1
(43) Pub. Date: Sep. 5, 2024

(54) LIPID NANOPARTICLE (LNP) DELIVERY SYSTEMS AND USES THEREOF

(71) Applicant: BOARD OF REGENTS, THE UNIVERSITY OF TEXAS SYSTEM, Austin, TX (US)

(72) Inventors: Hugh D.C. SMYTH, Austin, TX (US); Hairui ZHANG, Austin, TX (US); Debadyuti GHOSH, Austin, TX (US); Jasmim LEAL, Austin, TX (US); Melissa SOTO, Austin, TX (US); Robert O. WILLIAMS, III, Austin, TX (US)

(73) Assignee: BOARD OF REGENTS, THE UNIVERSITY OF TEXAS SYSTEM, Austin, TX (US)

(21) Appl. No.: 17/996,618

(22) PCT Filed: Apr. 20, 2021
(86) PCT No.: PCT/US2021/028199

§ 371 Date: Oct. 19, 2022

Related U.S. Application Data
(60) Provisional application No. 63/012,796, filed on Apr. 20, 2020.

(51) Int. Cl. A61K 9/127; A61K 9/00; A61K 9/51; A61K 31/713; C12N 15/88

(52) U.S. Cl. CPC A61K 9/1271; A61K 9/0075; A61K 9/0078; A61K 9/5123; A61K 31/713; C12N 15/88

(57) ABSTRACT

Lipid nanoparticles (LNPs) comprising biologically active poly nucleotides (e.g., RNAs) are provided. In some aspects, the LNP complexes are provided as aerosols and/or dry powders, such as for delivery to the lungs. Method of making and using such compositions are provided.

This application is a national phase application under 35 U.S.C. § 371 of International Application No. PCT/US2021/028199, filed Apr. 20, 2021, which claims the benefit of priority to U.S. Provisional Application No. 63/012,796, filed on Apr. 20, 2020, the entire contents of each of which are hereby incorporated by reference.

### BACKGROUND OF THE INVENTION

### 1. Field of the Invention

The present disclosure relates generally to the field of pharmaceutical formulation, biologics and the manufacture of the same. More particularly, Lipid nanoparticles and method of using the same to deliver nucleic acid molecules.

### 2. Description of Related Art

Delivery of nucleic acids through gene therapy and editing offers the promise of long-term correction and cure for genetic diseases including those affecting the lung, such as cystic fibrosis (CF). However, it is a challenge to systemically deliver gene therapies at sufficient therapeutic concentrations. Alternatively, directly delivering therapeutics to the lungs would be a more effective approach. Accordingly, pulmonary delivery of therapeutics has attracted growing attention due to the capability of the lungs to take up therapeutics for local deposition. However, there remains a significant need for compositions that can be used to efficiently and safely deliver nucleic acid-based therapeutics to tissues, such as lung tissue.

### SUMMARY OF THE INVENTION

In some embodiments, the present disclosure provides compositions comprising a biologically active polynucleotide molecules and a lipid nanoparticles (LNPs), wherein the LNPs comprises at least an ionizable lipid, at least a first phospholipid and at least a first PEG-lipid. In some aspects, the LNPs further comprise cholesterol. In some aspects, the biologically active polynucleotide molecules comprises RNA. In further aspects, the biologically active polynucleotide molecules comprises a mRNA. In still further aspects, the mRNA encodes therapeutic polypeptide or an antigen. In yet further aspects, the mRNA the therapeutic polypeptide is an enzyme. In other aspects, the mRNA encodes an antigen. In some aspects, the compositions further comprise an adjuvant. In further aspects, the adjuvant comprises alum. In some aspects, the biologically active polynucleotide molecules are encapsulated in the LNPs.

In some aspects, the LNPs comprise a molar ratio of ionizable lipid of about 0.1, 0.2, 0.3, 0.4, 0.5, 0.6, 0.7, 0.8, or 0.9, such as from about 0.4 to about 0.6. In some aspects, the LNPs comprise a molar ratio of PEG-lipid of about 0.01, 0.02, 0.03, 0.04, 0.05, 0.06, 0.07, 0.08, 0.09, or 0.1, such as from about 0.01 to about 0.05. In some aspects, the LNPs comprise a molar ratio of phospholipid of about 0.01, 0.02, 0.03, 0.04, 0.05, 0.1, 0.15, 0.2, 0.3, 0.4, or 0.5, such as from about 0.1 to about 0.2.In some aspects, the LNPs comprise a molar ratio of cholesterol of about 0.01, 0.02, 0.03, 0.04, 0.05, 0.1, 0.15, 0.2, 0.3, 0.4, or 0.5, such as from about 0.15 to about 0.49. In further aspects, the LNPs comprise a molar ratio of ionizable lipid of from about 0.4 to about 0.6; PEG-lipid of from about 0.01 to about 0.05; phospholipid of from about 0.1 to about 0.2 and cholesterol of from about 0.15 to about 0.49. In still further aspects, the phospholipid comprises DOPE; DSPC; and/or DPPC. In other aspects, the PEG lipid comprises DMG-PEG; DMPE-PEG; and/or DSPE-PEG. In still other aspects, the LNP comprises Dlin-MC3-DMA. In yet other aspects, the LNP comprises DOPE and DMG-PEG; DOPE and DMPE-PEG; DOPE and DPPC; DOPE and DSPE-PEG; DSPC and DMG-PEG; DSPC and DMPE-PEG; DSPC and DPPC; DSPC and DSPE-PEG; DPPC and DMG-PEG; DPPC and DMPE-PEG; DPPC and DPPC; or DPPC and DSPE-PEG.

In some aspects, the compositions further comprise a pH buffering agent. In some aspects, the LNPs comprise ionizable lipids, phospholipids, cholesterol, lecithin and/or poly-(ethylene) glycol (PEG)-lipid. In some aspects, the LNPs comprise cationic lipids; DOPE; DPPC; DSPC; DMPE-PEG; DMG-PEG; DSPE-PEG; Dlin-MC3-DMA; phospholipids; PEG-lipid and/or cholesterol. In some aspects, the LNPs have an average particle size of about 10 nm, 20 nm, 25 nm, 30 nm, 40 nm, 50 nm, 60 nm, 70 nm, 80 nm, 90 nm, 100 nm, 200 nm, 400 nm, 400 nm, 500 nm, 600 nm, 700 nm, 800 nm, 900 nm, or 1000 nm, such as between about 25 nm and 1000 nm, 50 nm and 1000 nm; 50 nm and 600 nm, or 80 nm and 200nm. In some aspects, the composition further comprises at least a first excipient. In further aspects, the first excipient comprises a sugar or sugar alcohol. In still further aspects, the first excipient comprises lactose, trehalose, sucrose, mannitol or sorbitol. In yet further aspects, the compositions comprise from about 5% to about 25% lactose, trehalose, sucrose, mannitol, or sorbitol.

In other embodiments, the present disclosure provides nebulized compositions in accordance with the compositions disclosed herein.

In still other embodiments, the present disclosure provides dry powder compositions in accordance with the compositions disclosed herein, said dry powder comprising at least a first excipient, said dry powder having been produced spray drying, spray freeze drying or freeze drying. In some aspects, the powder has a geometric particle size distribution Dv50, measured by dry Rodos method, of less than about 100 μm, 50 μm, 30 μm, 20 μm, 15 μm, or 12 um. In further aspects, the powder has a geometric particle size distribution Dv50, measured by dry Rodos method, of about 0.5 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, or 100 μm, such as from about 1 to 50 um or from about 3 to 50 um. In some embodiments, the powder has a density of about 0.5 g/cm³, 0.6 g/cm³, 0.7 g/cm³, 0.8 g/cm³, 0.9 g/cm³, 1.0 g/cm³, 1.1 g/cm³, 1.2 g/cm³, 1.3 g/cm³, 1.4 g/cm³, 1.5 g/cm³, 1.6 g/cm³, 1.7 g/cm³, 1.8 g/cm³, 1.9 g/cm³, 2.0 g/cm³, 2.1 g/cm³, 2.2 g/cm³, 2.3 g/cm³, 2.4 g/cm³, or 2.5 g/cm³, such as from about 1.0 to 2.0 g/cm³; 1.4 to 1.9 g/cm³; 1.4 to 1.9 g/cm³; or 1.5 to 1.7 g/cm³. In some aspects, the powder has a surface area of about 1 m²/g, 1.5 m²/g, 2 m²/g, 2.5 m²/g, 3 m²/g, 3.5 m²/g, 4 m²/g, 4.5 m²/g, 5 m²/g, 5.5 m²/g, 6 m²/g, 6.5 m²/g, 7 m²/g, 7.5 m²/g, 8 m²/g, 8.5 m²/g, 9 m²/g, 9.5 m²/g, or 10 m²/g, such as from about 2.0 to 8.5 m²/g; 2.0 to 7.5 m²/g; 3.0 to 7.5 m²/g; 2.0 to 5.0 m²/g; 2.5 to 4.5 m²/g; or 3.0 to 4.0 m²/g.

In some aspects, the first excipient comprises a sugar, or sugar alcohol. In further aspects, the sugar is a disaccharide. In some aspects, the first excipient comprises lactose, trehalose, sucrose, mannitol or sorbitol. In some aspects, the first excipient comprises at least about 50% of the powder by weight. In further aspects, the first excipient comprises about 50%-99.5%; 60%-99%; 70%-99%; 80%-99%; 90%-99% or 95%-99.5% of the powder by weight. In some aspects, the first excipient comprises a sugar, or sugar alcohol. In some aspects, the has a water content of less than 20%, 15% or 10%. In some aspects, the powder pharmaceutical composition has a water content of about 0.5% to 10%, 1% to 10%, 1.5% to 8% or 2% to 5%. In some aspects, the dry powder further comprises at least a second, third and/or fourth excipient. In further aspects, the second, third and/or fourth excipient comprises an amino acid or protein. In still further aspects, the second, third and/or fourth excipient comprises leucine or glycine. In some aspects, the second, third and/or fourth excipient comprises a polymer. In further aspects, the polymer comprises PEG, HPMC, PLGA, PVA, dextran, sodium alginate, chitosan, or PVP. In some aspects, the composition is formulated for use with an inhaler.

In yet other embodiments, the present disclosure provides inhalers comprising the compositions of the present disclosure. In some aspects, the inhaler is a single dose dry powder inhaler, a multi-dose dry powder inhaler, multi-unit dose dry powder inhaler, a metered dose inhaler, or a pressurized metered dose inhaler. In further aspects, the inhaler is a capsule-based inhaler. In some aspects, the inhaler is a low resistance inhaler. In other aspects, the inhaler is a high resistance inhaler. In some aspects, the inhaler is used with a flow rate of about L/min, 15 L/min, 20 L/min, 25 L/min, 50 L/min, 75 L/min, 100 L/min, 125 L/min, or 150 L/min, such as from about 10 L/min to about 150 L/min or from about 20 L/min to about 100 L/min.

In other embodiments, the present disclosure provides methods of treating a lung disease, lung injury or lung infection comprising administering an effective amount of a composition of the present disclosure to a subject. In some aspects, the lung disease is interstitial lung diseases, chronic obstructive pulmonary disease (COPD), asthma, cystic fibrosis (CF), pulmonary fibrosis or primary ciliary dyskinesia (PCD).

In still other embodiments, the present disclosure provides methods of stimulating an immune response in a subject comprising administering an effective amount of a composition of the present disclosure, wherein the biologically active polynucleotide molecules encode an antigen. In some aspects, the composition comprises LNPs and mRNA.

In yet other embodiments, the present disclosure provides methods of treating a disease in a subject comprising administering an effective amount of a composition of the present disclosure to the subject. In some aspects, the disease is a genetic disease.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows intracellular uptake of LNP formulations at different N/P ratios in HEK-293 cells measured by percent GFP expression (left axis) and fluorescence intensity (right axis).

FIGS. 2A-2D shows characterization of LNP formulations. (FIG. 2A) size, (FIG. 2B) zeta-potential, (FIG. 2C) encapsulation efficiency, and (FIG. 2D) pKa. Stability of the lipid nanoparticles was evaluated by measuring size and zeta-potential at day 1 and after 14 days from preparation and storage at 4° C. (mean±SD, n=3).

FIGS. 3A-3C show stability of LNP formulations before and after nebulization in terms of (FIG. 3A) size, (FIG. 3B) zeta-potential, and (FIG. 3C) encapsulation efficiency. The size (***p=0.0004) and encapsulation efficiency (****p<0.0001) of nebulized formulation were significantly different from pre-nebulized formulations.

FIGS. 4A & 4B show efficiency of intracellular uptake in HEK-293 cells over 16 days after LNPs preparation. (FIG. 4A) percent GFP expression, and (FIG. 4B) fluorescence intensity.

FIGS. 5A-5D show in vitro intracellular uptake in terms of percent GFP expression (FIGS. 5A & 5C) and fluorescence intensity (FIGS. 5B & 5D) of LNP formulations before and after nebulization in HEK-293 and NuLi-1 cells.

FIGS. 6A & 6B show efficacy and biodistribution of F2, F8, F11, F17 formulations with luciferase mRNA. (FIG. 6A) Efficacy of the four lead formulations before and after nebulization in lung as measured in total flux of luminescence 6 h after intratracheal delivery of 15 μg of total mRNA. (FIG. 6B) Representative images of the luciferase expression in lungs, heart, liver, and kidneys measured by IVIS imaging.

FIGS. 7A-7D show correlation between particle size and PEG-lipid. (FIG. 7A) Effect of PEG-lipid molar ratio on particle size before nebulization. (FIG. 7B) Effect of type of PEG-lipid on particle size before nebulization. (FIG. 7C) Effect of PEG-lipid molar ratio on particle size after nebulization. (FIG. 7D) Effect of type of PEG-lipid on particle size after nebulization.

FIGS. 8A-8D show correlation between zeta potential and PEG-lipid. (FIG. 8A) Significant effects of PEG-lipid molar ratio on zeta potential before nebulization. (FIG. 8B) Significant effects of type of PEG-lipid on zeta potential before nebulization. (FIG. 8C) Significant effects of PEG-lipid molar ratio on zeta potential after nebulization. (FIG. 8D) Significant effects of type of PEG-lipid on zeta potential after nebulization.

FIGS. 9A-9D show correlation of encapsulation efficiency and cholesterol molar ratio & type of phospholipid. (FIG. 9A) Significant correlation (p<0.05) between encapsulation efficiency and cholesterol molar ratio before nebulization. (FIG. 9B) No significant effects (p>0.05) of type of phospholipid on encapsulation efficiency before nebulization. (FIG. 9C) No significant correlation between encapsulation efficiency and cholesterol molar ratio after nebulization. (FIG. 9D) Significant effects of type of phospholipid on encapsulation efficiency after nebulization. **p<0.01.

FIGS. 10A-10F show correlation analysis between intracellular uptake (percent GFP expression and fluorescence intensity) and PEG-lipid molar ratio or type of phospholipid. (FIG. 10A) Significant effect of PEG-lipid molar ratio on percent GFP expression before nebulization. (FIG. 10B) Significant effect of type of phospholipid on percent GFP expression before nebulization. (FIG. 10C) Significant effect of PEG-lipid molar ratio on percent GFP expression before nebulization. (FIG. 10D) Significant effect of PEG-lipid molar ratio on percent GFP expression after nebulization. (FIG. 10E) No significant effect of type of phospholipid on percent GFP expression after nebulization. (FIG. 10F) Significant effect of PEG-lipid molar ratio on fluorescence intensity after nebulization.

FIGS. 11A-11H show orthogonal trends of intracellular uptake in terms of percent GFP expression and fluorescence intensity, whereby dotted line represented non-significance and solid line represented significance. (FIGS. 11A-11D): Correlation between intracellular uptake and formulation properties before nebulization. (FIGS. 11E-11H): Correlation between intracellular uptake and formulation properties after nebulization.

FIGS. 12A-12C show characterization of LNP formulations. (FIG. 12A) size, (FIG. 12B) zeta-potential, and (FIG. 12C) encapsulation efficiency.

FIGS. 13A-13D show in vitro intracellular uptake in terms of percent GFP expression (FIGS. 13A & 13B) and fluorescence intensity (FIGS. 13C & 13D) of LNP formulations in HEK-293 and NuLi-I cells.

### DESCRIPTION OF ILLUSTRATIVE EMBODIMENTS

### I. The Present Embodiments

Provided herein are lipid nanoparticles (LNP) carriers for biologically active nucleic acid molecules. In some cases, LNPs of the embodiments can comprise single stranded or double stranded RNA or DNA. Such polynucleotides can be encapsulated in or in complex with lipid nanoparticle. For example, in some cases, polynucleotides, such as mRNAs are provided in complex with LNPs. In certain aspects, the LNPs comprise at least one cationic lipid, at least one phospholipid, at least one PEGylated lipid and cholesterol. In certain aspects, the polynucleotide-LNP can be for gene replacement and/or gene editing. In some aspects, a mRNA-LNP complex can encode a therapeutically active protein (e.g., for gene replacement therapy) or an antigen (e.g., for vaccination). In preferred aspects, the LNP carriers and biologically active polynucleotides can be formulated as an aerosol (e.g., via nebulization) such as for delivery to lung. In further aspects, LNP complexes are provided in dry powders, such as by ultra-rapid freezing (URF). Preferably, such dry powder compositions additionally include at least a first excipient. For example, RNA-LNP powders are provided that further comprises at least a first excipient, such as sugar or amino acid. In some aspects, dry powders can be directly administered (e.g., by dispersion in the lungs) to subjects to treat a disease or stimulate an immune response.

### II. Formulation Preparation

In certain aspects, the present disclosure provides pharmaceutical compositions which may be prepared using spray drying, spray freeze drying, or freeze drying. In some cases, the methods employ an ultra-rapid freezing rate of up to 10,000 K/sec, e.g., at least 1,000, 2,000, 5,000 or 8,000 K/sec. In some embodiments, these methods involve dissolving the components of the pharmaceutical composition into a solvent to form a precursor solution. The solvents may be either water or an organic solvent. However, the in preferred aspects, the precursor solution is an aqueous solution that includes at least a first excipient and biologically active polynucleotide molecules. In some embodiments, the precursor solution may contain less than 10% w/v of the therapeutic agent and excipient. The precursor solution may contain less than 0.5%, 1%, 2%, 3%, 4%, 5%, 6%, 7%, 8%, 9%, or 10% w/v, or any range derivable therein.

This precursor solution may be deposited on a surface which is at a temperature that causes the precursor solution to freeze. In some embodiments, this temperature may be below the freezing point of the solution at ambient pressure. In other embodiments, a reduced pressure may be applied to the surface causing the solution to freeze at a temperature below the ambient pressure's freezing point. The surface may also be rotating or moving on a moving conveyer-type system thus allowing the precursor solution to distribute evenly on the surface. Alternatively, the precursor solution may be applied to surface in such a manner to generate an even surface.

After the precursor solution has been applied to the surface, the solvent may be removed to obtain a pharmaceutical composition. Any appropriate method of removing the solvent may be applied including evaporation under reduced pressure or elevated temperature or lyophilization. In some embodiments, the lyophilization may comprise a reduced pressure and/or a reduced temperature. Such a reduced temperature may be from 25° C. to about −200° C., from 20° C. to about −175° C., from about 20° C. to about −150° C., from 0° C. to about −125° C., from −20° C. to about −100° C., from −75° C. to about −175° C., or from −100° C. to about −160° C. The temperature is from about −20° C., −30° C., −35° C., −40° C., −45° C., −50° C., −55° C., −60° C., −70° C., −80° C., −90° C., −100° C., −110° C., −120° C., −130° C., −140° C., −150° C., −160° C., −170° C., −180° C., −190° C., to about −200° C., or any range derivable therein. Additionally, the solvent may be removed at a reduced pressure of less than 500 mTorr, 450 mTorr, 400 mTorr, 375 mTorr, 350 mTorr, 325 mTorr, 300 mTorr, 275 mTorr, 250 mTorr, 225 mTorr, 200 mTorr, 175 mTorr, 150 mTorr, 125 mTorr, 100 mTorr, 75 mTorr, 50 mTorr, or 25 mTorr.

Such as composition prepared using these methods may exhibit a brittle nature such that the composition is easily sheared into smaller particles when processed through a device. These compositions have high surface areas as well as exhibit improved flowability of the composition. Such flowability may be measured, for example, by the Carr index or other similar measurements. In particular, the Carr's index may be measured by comparing the bulk density of the powder with the tapped density of the powder. Such compounds may exhibit a favorable Carr index and may result in the particles being better sheared to give smaller particles when the composition is processed through a secondary device to further process a powder composition.

### III. Components of Compositions of the Embodiments cl A. Composition Including Biologically Active Polynucleotides

Methods and composition of the embodiments concern biologically active polynucleotides. In some cases, these can comprise single stranded or double stranded RNA or DNA. Such polynucleotides can be encapsulated in or in complex with nanoparticles. For example, in some cases polynucleotides, such as mRNAs are provided in complex with LNPs. For example, the mRNA may encode a therapeutic polypeptide or an antigen. In some aspects, mRNA molecules comprise a 5′ cap; a 5′ UTR; a 3′UTR; and/or a poly-A tail. mRNA molecules can provide a more direct method of expressing a polypeptide of interest in a target cell. However, such molecules are typically highly liable and rapidly degraded. However, in some aspects, LNP and/or processing according to the embodiments can be used to substantially stabilize mRNA. In prefer aspects, mRNA is provided encapsulated in or in complex with LNPs.

In some aspects, a nucleic acid molecule of the embodiments encodes a therapeutic polypeptide. For example, the therapeutic protein may be a protein, such as an enzyme that is non-functional or disrupted in a particular disease state (e.g., CFTR in cystic fibrosis).

In further aspects, a polynucleotide of the embodiments encodes an antigen, such as an antigen from a pathogen or a cancer cell-associated antigen. For example, the cancer associated antigen can be CD19, CD20, RORI, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, GD2, CD123, CD33, CD138, CD23, CD30 , CD56, c-Met, mesothelin, GD3, HERV-K, IL-11Ralpha, kappa chain, lambda chain, CSPG4, ERBB2, EGFRvIII or VEGFR2. In some specific aspects the antigen is GP240, 5T4, HER1, CD-33, CD-38, VEGFR-1, VEGFR-2, CEA, FGFR3, IGFBP2, IGF-1R, BAFF-R, TACI, APRIL, Fn14, ERBB2 or ERBB3

Antigens useful in the present disclosure may include those derived from viruses including, but not limited to, those from the family Arenaviridae (e.g., Lymphocytic choriomeningitis virus), Arterivirus (e.g., Equine arteritis virus), Astroviridae (Human astrovirus 1), Birnaviridae (e.g., Infectious pancreatic necrosis virus, Infectious bursal disease virus), Bunyaviridae (e.g., California encephalitis virus Group), Caliciviridae (e.g., Caliciviruses), Coronaviridae (e.g., Human coronaviruses 299E and OC43), Deltavirus (e.g., Hepatitis delta virus), Filoviridae (e.g., Marburg virus, Ebola virus), Flaviviridae (e.g., Yellow fever virus group, Hepatitis C virus), Hepadnaviridae (e.g., Hepatitis B virus), Herpesviridae (e.g., Epstein-Bar virus, Simplexvirus, Varicellovirus, Cytomegalovirus, Roseolovirus, Lymphocryptovirus, Rhadinovirus), Orthomyxoviridae (e.g., Influenzavirus A, B, and C), Papovaviridae (e.g., Papillomavirus), Paramyxoviridae (e.g., Paramyxovirus such as human parainfluenza virus 1, Morbillivirus such as Measles virus, Rubulavirus such as Mumps virus, Pneumovirus such as Human respiratory syncytial virus), Picornaviridae (e.g., Rhinovirus such as Human rhinovirus 1A, Hepatovirus such Human hepatitis A virus, Human poliovirus, Cardiovirus such as Encephalomyocarditis virus, Aphthovirus such as Foot-and-mouth disease virus O, Coxsackie virus), Poxyiridae (e.g., Orthopoxvirus such as Variola virus or monkey poxvirus), Reoviridae (e.g., Rotavirus such as Groups A-F rotaviruses), Retroviridae (Primate lentivirus group such as human immunodeficiency virus 1 and 2), Rhabdoviridae (e.g., rabies virus), Togaviridae (e.g., Rubivirus such as Rubella virus), Human T-cell leukemia virus, Murine leukemia virus, Vesicular stomatitis virus, Wart virus, Blue tongue virus, Sendai virus, Feline leukemia virus, Simian virus 40, Mouse mammary tumor virus, Dengue virus, HIV-1 and HIV-2, West Nile, HINI, SARS, 1918 Influenza, Tick-borne encephalitis virus complex (Absettarov, Hanzalova, Hypr), Russian Spring-Summer encephalitis virus, Congo-Crimean Hemorrhagic Fever virus, Junin Virus, Kumlinge Virus, Marburg Virus, Machupo Virus,

Kyasanur Forest Disease Virus, Lassa Virus, Omsk Hemorrhagic Fever Virus, FIV, SIV, Herpes simplex 1 and 2, Herpes Zoster, Human parvovirus (B19), Respiratory syncytial virus, Pox viruses (all types and serotypes), Coltivirus, Reoviruses-all types, and/or Rubivirus (rubella).

Antigens useful in the present disclosure may include those derived from bacteria including, but not limited to, Streptococcus agalactiae, Legionella pneumophilia, Streptococcus pyogenes, Escherichia coli, Neisseria gonorrhosae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis, Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Candida albicans, Cryptococcus neoformans, Histoplasma capsulatum, Coccidioides immitis, Blastomyces dermatitidis, Aspergillus fumigatus, Penicillium marneffei, Bacillus anthracis, Bartonella, Bordetella pertussis, Brucella—all serotypes, Chlamydia trachomatis, Chlamydia pneumoniae, Clostridium botulinum—-anything from clostridium serotypes, Haemophilus influenzae, Helicobacter pylori, Klebsiella—all serotypes, Legionella—all serotypes, Listeria, Mycobacterium—all serotypes, Mycoplasma—human and animal serotypes, Rickettsia—all serotypes, Shigella—all serotypes, Staphylococcus aureus, Streptococcus—S. pneumoniae, S. pyogenes, Vibrio cholera, Yersinia enterocolitica, and/or Yersinia pestis.

Antigens useful in the present disclosure may include those derived from parasites including, but not limited to, Ancylostomahuman hookworms, Leishmania—all strains, Microsporidium, Necator human hookworms, Onchocerca filarial worms, Plasmodium—all human strains and simian species, Toxoplasma—all strains, Trypanosoma—all serotypes, and/or Wuchereria bancrofti filarial worms.

### B. Nanoparticle and nanoparticle complexes

As used herein, the term “nanoparticle” refers to any material having dimensions in the 1-1,000 nm range. In some embodiments, nanoparticles have dimensions in the 50-500 nm range. Nanoparticles used in the present embodiments include such nanoscale materials as a lipid-based nanoparticle, a superparamagnetic nanoparticle, a nanoshell, a semiconductor nanocrystal, a quantum dot, a polymer-based nanoparticle, a silicon-based nanoparticle, a silica-based nanoparticle, a metal-based nanoparticle, a fullerene and a nanotube (Ferrari, 2005). The conjugation of polypeptide or nucleic acids to nanoparticles provides structures with potential application for targeted delivery, controlled release, enhanced cellular uptake and intracellular trafficking, and molecular imaging of therapeutic peptides in vitro and in vivo (West, 2004; Stayton et al., 2000; Ballou et al., 2004; Frangioni, 2003; Dubertret et al., 2002; Michalet et al., 2005; Dwarakanath et al., 2004.

### (2) Lipid nanoparticles (LNPs)

Lipid-based nanoparticles include liposomes, lipid preparations and lipid-based vesicles (e.g., DOTAP:cholesterol vesicles). Lipid-based nanoparticles may be positively charged, negatively charged or neutral. In certain embodiments, the lipid-based nanoparticle is neutrally charged (e.g., a DOPC liposome).

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes and multivesicular liposomes. Liposomes provided herein may be positively charged, negatively charged or neutrally charged. In certain embodiments, the liposomes are neutral in charge.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a polypeptide or nucleic acids may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. No. 5,030,453, and U.S. Pat. No. 6,680,068, all of which are hereby incorporated by reference in their entirety without disclaimer. A process of making liposomes is also described in WO04/002453A1. Neutral lipids can be incorporated into cationic liposomes (e.g., Farhood et al., 1995). Various neutral liposomes which may be used in certain embodiments are disclosed in U.S. Pat. No. 5,855,911, which is incorporated herein by reference. These methods differ in their respective abilities to entrap aqueous material and their respective aqueous space-to-lipid ratios.

The size of a liposome varies depending on the method of synthesis. Liposomes in the present embodiments can be a variety of sizes. In certain embodiments, the liposomes are small, e.g., less than about 100 nm, about 90 nm, about 80 nm, about 70 nm, about 60 nm, or less than about 50 nm in external diameter. For example, in general, prior to the incorporation of nucleic acid, a DOTAP:cholesterol liposome for use according to the present embodiments comprises a size of about 50 to 500 nm. Such liposome formulations may also be defined by particle charge (zeta potential) and/or optical density (OD). For instance, a DOTAP:cholesterol liposome formulation will typically comprise an OD₄₀₀ of less than 0.45 prior to nucleic acid incorporation. Likewise, the overall charge of such particles in solution can be defined by a zeta potential of about 50-80 mV.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

In certain embodiments, the lipid based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes.

Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DSSP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

Phospholipids may be from natural or synthetic sources. However, phospholipids from natural sources, such as egg or soybean phosphatidylcholine, brain phosphatidic acid, brain or plant phosphatidylinositol, heart cardiolipin and plant or bacterial phosphatidylethanolamine are not used, in certain embodiments, as the primary phosphatide (i.e., constituting 50% or more of the total phosphatide composition) because this may result in instability and leakiness of the resulting liposomes.

### C. Excipients

In some aspects, the present disclosure comprises one or more excipients formulated into pharmaceutical compositions. In some embodiments, the excipients used herein are water soluble excipients. These water soluble excipients include saccharides such as disaccharides such as sucrose, trehalose, or lactose, a trisaccharide such as fructose, sucrose, glucose, glacatose, or raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In some aspects, the present pharmaceutical compositions may further exclude a hydrophobic or waxy excipient such as waxes and oils. Some non-limiting examples of hydrophobic excipients include hydrogenated oils and partially hydrogenated oils, palm oil, soybean oil, castor oil, carnauba wax, beeswax, palm wax, white wax, castor wax, or lanoline. Additionally, the present disclosure may further comprise one or more amino acids or an amide or ester derivative thereof. In some embodiments, the amino acids used may be one of the 20 canonical amino acids such as glycine, alanine, valine, isoleucine, leucine, methionine, phenylalanine, tyrosine, tryptophan, serine, threonine, asparagine, glutamine, cysteine, selenocysteine, proline, arginine, histidine, lysine, aspartic acid, or glutamic acid. These amino acids may be in the D or L orientation or the amino acids may be an α-, β-, γ-, or δ-amino acids. In other embodiments, one of the common non-canonical amino acids may be used such as carnitine, GABA, carboxyglutamic acid, levothyroxine, hydroxyproline, seleonmethionine, beta alanine, ornithine, citrulline, dehydroalanine, δ-aminolevulinic acid, or 2-aminoisobutyric acid. Additional excipients, especially compositions prepared using freeze drying, that may be used in these compositions include those described in Ferrati et al. (Ferrati et al., 2018). These excipients include sucrose, mannitol, histidine buffer, and polysorbate 80.

In some aspects, the amount of the excipient in the precursor solution for making a powder composition is from about 0.5% to about 20% w/w, from about 1% to about 10% w/w, from about 2% to about 8% w/w, or from about 2% to about 5% w/w. The amount of the excipient in the precursor solution comprises from about 0.5%, 0.75%, 1%, 1.25%, 1.5%, 1.75%, 2%, 2.5%, 3%, 3.5%, 4%, 4.5%, 5%, 6%, 7%, 8%, 9%, to about 10% w/w, or any range derivable therein. In one embodiment, the amount of the excipient in a dry powder of the embodiments is about 10% to 99.5% w/w of the total weight of the pharmaceutical composition, such as about 50% to 99%, 75% to 99% or 80% to 98%.

### III. Definitions

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

As used herein, the term “nanoparticle” has its customary and ordinary definition and refers to discrete particles which behave as a whole unit rather than as individual molecules within the particle. A nanoparticle may have a size from about 1 to about 10,000 nm with ultrafine nanoparticles having a size from 1 nm to 100 nm, fine particles having a size from 100 nm to 2,500 nm, and coarse particles having a size from 2,500 nm to 10,000 nm. In some embodiments, the nanoaggregates described herein may comprise a composition of multiple nanoparticles and have a size from about 10 nm to about 100 μm.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

### IV. Examples

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1

Development of Lipid Nanoparticles through Design of Experiments for Aerosolized Pulmonary Delivery of mRNA

### A. Material and Methods

### 1. Materials

DLin-MC3-DMA was purchased from Biofine International Inc., Vancouver, BC. 1,2-dipalmitoyl-sn-glycero-3-phosphocholine (DPPC), 1,2-dimyristoyl-rac-glycero-3-methoxypolyethylene glycol-2000 (DMG-PEG-2000), 1,2-distearoyl-sn-glycero-3-phosphocholine (DSPC), 1,2-distearoyl-sn-glycero-3-phosphoethanolamine-N-[Amino (Polyethylene Glycol) 2000 (DSPE-PEG 2000), and (Delta 9 cis)/1,2-dioleoyl-sn-glycero-3-phosphoethanolamine (DOPE) were purchased from Avanti Polar Lipids, AL, USA. N-(methylpolyoxyethyleneoxycarbonyl)-1,2-dimyristoyl-sn-glycero-3-phosphoehtanolamine (DMPE-PEG 2000) was purchased from NOF Corporation, Tokyo, JP. Cholesterol was purchased from Sigma Aldrich, MO. Ethanol (molecular grade) was purchased from Decon Laboratories, Inc., PA. CleanCap® Enhanced Green Fluorescent Protein (EGFP) mRNA and CleanCap® Firefly luciferase (FLuc) mRNA were purchased from TriLink, San Diego, CA, USA. Slide-A-Lyzer™ Gamma Irradiated Dialysis Cassette (10 kDa), Quanit-iT™ RiboGreen® RNA Reagent and Kit (Invitrogen), and Opti-MEM™ Reduced Serum Media (Gibco) were purchased from ThermoFisher Scientific Inc., Waltham, MA, USA. Dulbecco's

Modification of Eagle's Medium (DMEM), Fetal Bovine Serum (FBS), and Penicillin/Streptomycin (100X) were purchased from Corning, Manassas, VA, USA. Balb/c mice were purchased from Charles River Laboratories, Inc, Wilmington, MA, USA.

### 2. Methods

Preparation of LNP formulations. Lipid nanoparticles containing EGFP mRNA or FLuc mRNA were prepared by combining an aqueous phase (mRNA diluted in 100 mM sodium acetate citrate buffer, pH 3.0) and an organic phase containing ethanol and lipids according to each formulation (Table 2) using a microfluidic mixer (Precision Nanosystems, Canada; Leung et al., 2015). After preparation, LNP formulations were dialyzed into 1X PBS (pH 7.4) for 2 hours in 10K MWCO Slide-A-Lyzer dialysis cassettes (Thermo Fisher Scientific, MA).

Measurements of size and zeta potential. The size and zeta potential of LNP formulations were characterized by using Zetasizer Nano-ZS (Malvern Instruments MA). Each formulation was 10-fold diluted in 0.1X PBS buffer for size measurement and 40-fold diluted in 0.1X PBS for zeta potential measurement. Dynamic light scattering was performed on diluted samples at 25° C. with 173° and the reported z-average diameter is a mean of three measurements.

mRNA Encapsulation efficiency. mRNA encapsulation efficiency was evaluated by low range Quanti-iT RiboGreen RNA reagent assay (Thermo Fisher Scientific, MA). Each LNP sample was diluted into TE buffer down to a mRNA concentration of 0.2 ng/μL. Aliquots of each LNP working solution was further diluted 1:1 in TE buffer (measuring unencapsulated mRNA) or 1:1 in TE buffer with 4% Triton-X100 (measuring total mRNA-both encapsulated within LNPs and unencapsulated free mRNA) in a 96-well plate. Samples were prepared in duplicate and 100 μL of 2000-fold diluted Quanti-iT™ RiboGreen RNA reagent was added to each sample the fluorescence intensity was measured by plate reader at excitation and emission wavelengths of 480 and 520 nm (Infinite M200, Tecan, Switzerland), respectively.

TNS assays. A series of buffers with pH ranging from 2.5 to 11 (pH 2.5, pH 3, pH 3.5, pH 4, pH 4.6, pH 5, pH 5.5, pH 5.8, pH 6, pH 6.5, pH 7, pH 7.5, pH 8, pH 8.5, pH 9, pH 9.5, pH 10, pH 10.5, pH 11) were prepared by adjusting the pH of a buffer solution consisting of 10 mM HEPES, 10 mM MES, 10 mM ammonium acetate, 130 mM NaCl with 1 N HCl. Also, 90 μL of each buffer solution was added to a 96-well plate. 2 μL of TNS stock solution (300 μM in DMSO) was added to the buffer solutions at different pH in the 96-well plate. Then 3 μL of an LNP solution (prepared with mRNA) was added to the above mixture. The fluorescence intensity was measured at an excitation wavelength of 325 nm and an emission wavelength of 435 nm. The fluorescence intensity was plotted against pH values and fitted using a three-parameter logistic equation (GraphPad Prism v.6, GraphPad Software). The pH value at which half of the maximum fluorescence is reached was regarded as the pKa of LNP formulations.

Aerosolization of LNP formulations. It has been shown that vibrating mesh nebulizers can be used to aerosolize shear susceptible formulations such as liposomes and niosomes and therefore are a good alternative to air jet and ultrasound nebulizers (Wagner et al., 2006; Elhissi et al., 2013). LNP formulations were added to the Aerogen Solo (Aerogen Ltd, Galway Ireland), which is a vibrating mesh nebulizer and the aerosol was subsequently collected by condensation in precooled tubes.

Cell culture. HEK-293 cells were cultured with Dulbecco's Modified Eagle Medium containing 10% FBS and 1% penicillin streptomycin. NuLi-1 cells (ATCC CRL-4013) were cultured in flasks pre-coated with 60 μg/mL solution of human placental collagen type IV (Sigma Aldrich, MO) and grown in bronchial epithelial growth medium (BEGM) supplemented with SingleQuot additives from Lonza (BEGM Bullet Kit, reference CC-3170) and 50 μg/mL G-418. All cell lines were maintained as monolayer cultures at 37° C. and 5% CO₂.

Intracellular uptake In vitro. Cells were seeded in 96-well plates at a cell density of 12,500 cells/well and grown for 24 hours at 37° C. and 5% CO₂. Then 10 μL of LNP at a 10 ng EGFP mRNA/μL concentration was added to cells in 0.2 mL cell culture media for 24 hours. After, the cell culture media was removed, and cells were washed with 1X PBS. To detach the cells, 100 uL of 0.25% trypsin-EDTA solution was added to each well and incubated at 37° C. for 8-10 minutes. Next, 100 μL of 1% FBS in Dulbecco's phosphate buffered saline was added, cells were spun at 125 x g for 5 to 10 minutes and the supernatant was discarded. Cells were resuspended in 50 μL 1X PBS with 0.25 μL propidium iodide (PI) (1 mg/mL) solution. Cell percent GFP expression (i.e. transfection efficiency) and fluorescence intensity were analyzed by flow cytometry.

In vivo transfection. All animal protocols were approved by the Institutional Animal Care and Use Committee at the University of Texas at Austin. Balb/c mice (female, 6-8 weeks) were anesthetized under a continuous flow of 2% isoflurane, and approximately 50 μL of LNP containing 1.5 μg of FLuc mRNA/uL in PBS were administered intratracheally. After 6 hours, mice were intraperitoneally (i.p.) injected with D-Luciferin solution (30 mg/ml) to reach 150 mg Luciferin/kg body weight. After 15 minutes, mice were sacrificed and the lungs were carefully harvested and imaged by an In Vivo Imaging System (IVIS), with bioluminescence setting and a luminescent exposure time of 60 sec. Quantification of luminescence (in radiance [p/sec/cm²/sr]) was performed with Living Image 4.3 software (PerkinElmer).

Statistical analysis. The statistical analysis was performed using JMP 13. Data values are expressed as mean±standard deviations (SD). When required, one-way analysis of variance (one-way ANOVA) or Student's t-test was performed. *p-values≤0.05, **p-values≤0.01, ***p-values≤0.001, and ****p-values≤0.0001 were considered statistically significant.

### B. Results and Discussion

### 1. Results

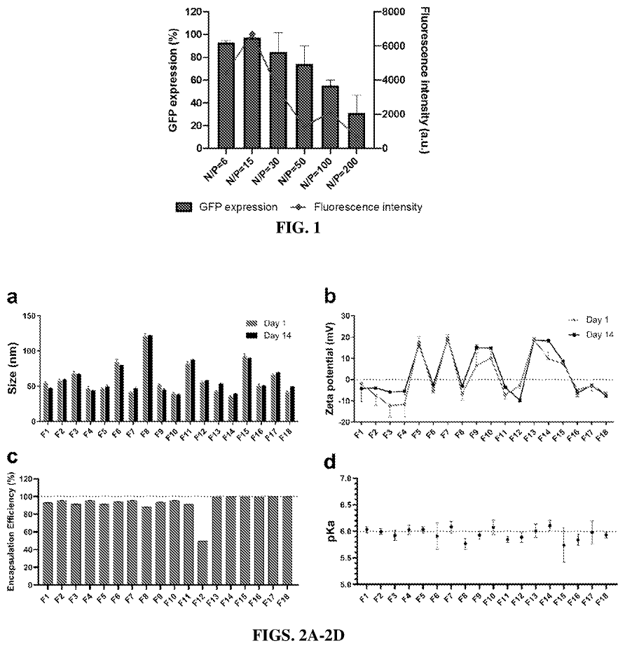

Effects of N/P ratio on the efficacy of LNP formulations. To investigate the effects of the N/P ratio on intracellular uptake, six LNP formulations encapsulating EGFP mRNA were prepared by varying the N/P ratio between 6 to 200. LNP formulations were composed of DLin-MC3-DMA, a phosphatidylcholine (1,2-distearoyl-sn-glycero-3-phosphocholine, DSPC), cholesterol, and a PEG-lipid (polyethylene glycol-dimyristolglycerol, PEG-DMG) at a single molar ratio of 50:10:38.5:1.5, respectively (as previously described in Jayaraman et al., 2012). The different N/P ratios (N/P=6, 15, 30, 50, 100, and 200) were achieved by varying the relative amount of lipid composition added to the mRNA (10 ng/μl). LNPs were prepared and the intracellular uptake of each formulation was evaluated in HEK-293 cells by flow cytometry. As shown in FIG. 1, the LNP formulation with an N/P ratio=15 demonstrated both the highest percent GFP expression and mean fluorescence intensity. The intracellular uptake decreased as the N/P ratio increased from 15 to 200. The N/P ratio=15 was maintained for the following experiments, and this particular formulation is subsequently used in the experiments as the “reference formulation”.

DOE: Mixture experimental design with constraints. LNPs consist generally of four lipid components: ionizable lipid, phospholipid, PEG-lipid, and cholesterol. The different types and amount of lipids may affect the transfection efficacy of LNP formulations (Kauffman et al., 2015). One-factor-at-a-time design methods have been employed in several studies to investigate the effect of formulation composition on the efficacy of each LNP formulation (Belliveau et al., 2012; Akinc et al., 2009). However, this approach does not account for potential second-order interactions between composition parameters, which makes it less desirable for optimization of LNP formulations. Alternatively, fractional factorial design has been used to maximize the potency of LNP formulations for mRNA delivery (Kauffman et al., 2015). Although this method investigates second-order effects, the fact that not all variables can be included in the design is a major limitation. In order to systematically investigate the effects of variables on the potency of LNP formulations, a mixture design with constraints was employed in this study (Table 2). Using JMP software, a design of 18 LNP formulations was generated for testing (Table 3).

TABLE 2
Limits of experimental design space.

| Component | Lower limit (molar ratio) | Upper limit (molar ratio) |
|---|---|---|
| Ionizable lipid | 0.4 | 0.6 |
| Phospholipid | 0.1 | 0.2 |
| PEG-lipid | 0.01 | 0.05 |
| Cholesterol | 0.15 | 0.49 |

TABLE 3
Composition of LNP formulations.

| | | | Molar composition | | | |
|---|---|---|---|---|---|---|
| Formulation | | | Dlin-MC3- | | | |
| # | Phospholipid | PEG-lipid | DMA | Phospholipid | PEG-lipid | Cholesterol |
| 1 | DOPE | DMG-PEG | 0.6 | 0.2 | 0.05 | 0.15 |
| 2 | DOPE | DMPE-PEG | 0.4 | 0.2 | 0.01 | 0.39 |
| 3 | DSPC | DMG-PEG | 0.5 | 0.14 | 0.01 | 0.35 |
| 4 | DOPE | DMPE-PEG | 0.6 | 0.15 | 0.03 | 0.22 |
| 5 | DSPC | DSPE-PEG | 0.4 | 0.2 | 0.05 | 0.35 |
| 6 | DPPC | DMG-PEG | 0.4 | 0.1 | 0.01 | 0.49 |
| 7 | DPPC | DSPE-PEG | 0.4 | 0.1 | 0.05 | 0.45 |
| 8 | DPPC | DMG-PEG | 0.6 | 0.2 | 0.01 | 0.19 |
| 9 | DOPE | DSPE-PEG | 0.6 | 0.1 | 0.05 | 0.25 |
| 10 | DOPE | DSPE-PEG | 0.4 | 0.2 | 0.03 | 0.37 |
| 11 | DPPC | DMPE-PEG | 0.6 | 0.2 | 0.01 | 0.19 |
| 12 | DSPC | DMG-PEG | 0.6 | 0.2 | 0.05 | 0.15 |
| 13 | DSPC | DSPE-PEG | 0.5 | 0.1 | 0.05 | 0.35 |
| 14 | DOPE | DSPE-PEG | 0.4 | 0.15 | 0.05 | 0.4 |
| 15 | DPPC | DSPE-PEG | 0.6 | 0.1 | 0.01 | 0.29 |
| 16 | DSPC | DMPE-PEG | 0.5 | 0.2 | 0.03 | 0.27 |
| 17 | DOPE | DMG-PEG | 0.4 | 0.16 | 0.01 | 0.43 |
| 18 | DPPC | DMPE-PEG | 0.4 | 0.1 | 0.03 | 0.47 |

Characterization of mRNA-LNPs. Based on a mixture design with constraints, 18 formulations with an N/P ratio=15 were prepared using the NanoAssemblr® benchtop system. The size and zeta potential of the LNPs were evaluated by DLS. As shown in FIG. 2A, the particle size of the LNP formulations before nebulization varied from 35.7 +1.1 nm (F14) to 120.9 +3.4 nm (F8), while the zeta potential ranged from-12.2 +5.5 mV (F3) to 18.8 +1.2 mV (F13) (FIG. 2B). Furthermore, the size and zeta potential of the LNP formulations did not show significant changes after 14 days of storage in 4° C., which indicated that the size and surface charge of all formulations remained stable for at least 2 weeks (FIGS. 2A & 2B). The encapsulation efficiency of the formulations was evaluated by RiboGreen assay. Most of the formulations possessed a high encapsulation efficiency greater than 80%, except for F12 which showed 49% encapsulation efficiency (FIG. 2C). It has been previously reported that the pKa of LNPs may be critical for endosomal escape and has been implicated as a correlator for in vivo efficacy of gene therapy (Jayaraman et al., 2012). Therefore, the pKa of LNP formulations loaded with EGFP mRNA was measured using the TNS assay, and the pKa ranged from 5.74 (F15) to 6.11 (F14) (FIG. 2D).

To translate LNP formulations for clinical use, they must be able to be aerosolized for pulmonary delivery without significant instability. Towards that end, it was investigated the effects of nebulization on the LNP formulations and subsequently identified the formulations that retained high intracellular uptake in vitro following nebulization. LNP formulations were aerosolized by the Aerogen Solo nebulizer and the potency of each nebulized formulation was evaluated in human embryonic kidney HEK-293 and human bronchial epithelial NuLi-1 cell lines. After nebulization, the size of the LNP formulations ranged from 100.9 nm (F12) to 1480.7 nm (F7) and showed a significant increase compared to the pre-nebulized LNP formulations, while the zeta potential showed no significant changes amongst all formulations (FIGS. 3A-3C). It is worth noting that F8 had the smallest change in size upon nebulization, and F7 showed the largest change in size after nebulization. The encapsulation efficiency of the LNP formulations significantly decreased after nebulization, which indicated that the mRNA potentially leaked from the LNPs upon the nebulization process. The encapsulation efficiency of nebulized LNP formulations ranged from 15.5% (F12) to 79.9% (F17).

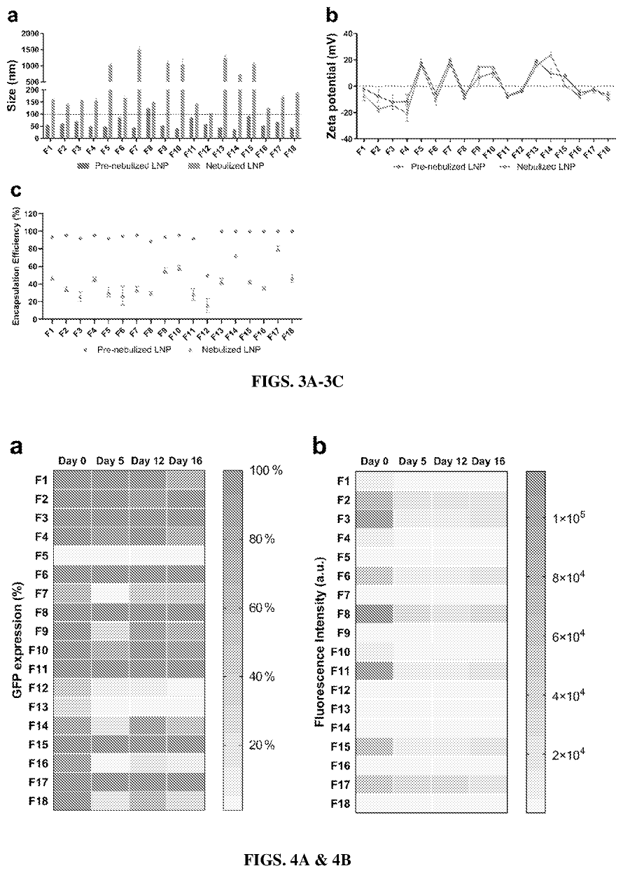

Intracellular uptake of LNP formulations in HEK-293 and NuLi-1 cells. The intracellular uptake of pre-and post-nebulized LNP mRNA formulations was assessed using flow cytometry by measuring percent GFP expression and fluorescence intensity in HEK-293 and NuLi-1 cell lines. On day 0 (i.e. incubation same day as preparation of formulations), the intracellular uptake of each mRNA-encapsulated formulation was measured in HEK-293 cells to identify formulations that exhibited higher transfection than the reference formulation (DLin-MC3-DMA:DSPC:cholesterol:PEG-DMG=50:10:38.5:1.5, N/P=15). It was found that most formulations showed over 50% GFP expression, except F5, F12, and F13. Notably, although most formulations had relatively high percent GFP expression, the intracellular uptake in terms of fluorescence intensity varied among the formulations. Eight out of 18 formulations (F2, F3, F4, F6, F8, F11, F15 and F17) showed a significantly higher fluorescence intensity compared to the reference formulation, which showed a mean fluorescence intensity of 6708 a.u. in HEK-293 cells on Day 0. The percent GFP expression of these eight formulations were as high as over 95% and showed no significant differences when compared to the reference formulation. Next, the stability of LNPs (i.e. lack of premature mRNA leakage) was tested by quantifying their intracellular uptake after 0, 5, 12, and 16 days of refrigerated storage. As shown in FIG. 4A, eight formulations (F2, F3, F6, F8, F10, F11, F15 and F17) remained stable in terms of percent GFP expression after 16 days of storage at 4° C. In contrast, the fluorescence intensity of all formulations decreased significantly after 5 days of storage at 4° C. (FIG. 4B). Specifically, F2, F3, F6, F8, F11, F15, and F17 showed a fluorescence intensity of over 18,000 a.u. which were significantly higher than the reference formulation after 16 days of storage.

Upon nebulization, all LNP formulations showed significantly decreased fluorescence intensity compared to pre-nebulized LNP formulations in both HEK-293 cells and NuLi-1 cells. This finding indicates that the aerosolization process negatively affected the mRNA transfection in vitro. It was found that F2, F3, F8, F11, and F17 showed no significant changes in terms of percent GFP expression after nebulization compared to pre-nebulized LNPs (FIGS. 5A & 5C). Despite a significant decrease of fluorescence intensity observed in all LNP formulations, the aforementioned five formulations retained relatively high fluorescence intensity (over 3000 a.u.) upon nebulization (FIGS. 5B & 5D). In NuLi-1 cells, although F2, F8, F11, and F17 showed decreased percent GFP expression and fluorescence intensity upon nebulization, these four formulations still demonstrated relatively high GFP expression (over 50%) and fluorescence intensity (over 1000 a.u.) compared to other formulations. In summary, four formulations (F2, F8, F11 and F17) with relatively high intracellular uptake after 16 days of storage and nebulization were identified.

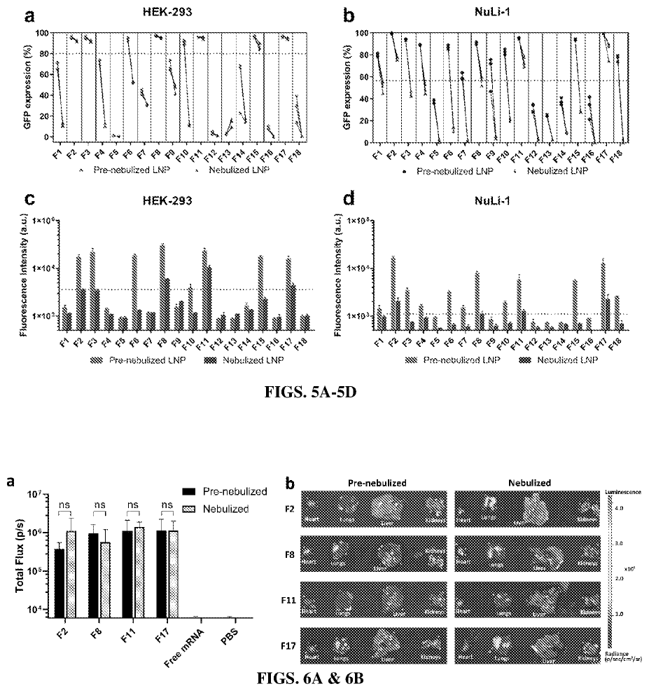

Intratracheal delivery of LNP formulations to mice. Based on intracellular uptake in vitro, four lead formulations (F2, F8, F11 and F18) were selected for further study in vivo. Specifically, firefly luciferase (Luc) mRNA was loaded into these LNP formulations and nebulized by an Aerogen Solo nebulizer. The collected nebulized dispersions were compared to pre-nebulized controls using intratracheal instillation administration to lungs of mice to investigate in vivo transfection and biodistribution. After 6 h post-administration, luciferase activity was predominantly detected in the lung compared to other organs for the four lead formulations, irrespective of the nebulization process (FIG. 6). Interestingly, there was no statistically significant difference in luminescence intensity between mice dosed with either pre-nebulized or nebulized LNP formulations, which indicated that the candidate formulations retained their function after nebulization.

### 2. Discussion

This work highlights a DOE approach to discover LNP formulations that are suitable for aerosolized delivery of mRNA. Using DOE, 18 formulations of various lipid compositions were prepared and characterized in terms of physicochemical properties and intracellular uptake. Four lead formulations that had relatively higher intracellular uptake before and after nebulization were identified and intratracheally delivered to mice, where they showed the ability to deliver mRNA to lungs in vivo before and after nebulization. Extensive statistical analysis of formulations helped identify certain parameters that impacted stability and intracellular delivery of nanoparticles.

Composition of LNP formulations influenced their physicochemical properties (size, zeta potential, and encapsulation efficiency) before and after nebulization. It was found that pre-nebulized dispersions had a particle size that was dependent on the molar ratio of PEG-lipid used. In these pre-nebulized formulations, it appeared that the type of PEG-lipid used did not influence particle size in a significant way. In contrast, the nebulized dispersions were significantly influenced by the type of PEG-lipid used in the formulation. These observations are discussed below.

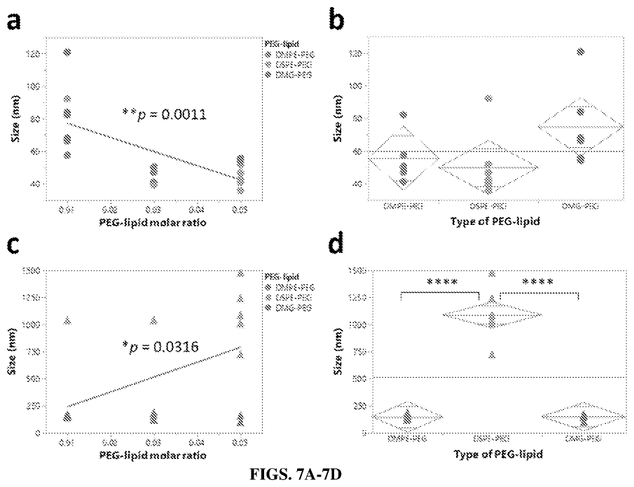

To explore the correlation between LNP size and each LNP component, the size of the LNPs before and after nebulization was plotted against each component, and the orthogonal trend was analyzed. A statistically significant (p<0.05) trend of decreasing size was observed with increasing molar PEG-lipid composition for pre-nebulized LNP formulations, independent of the other formulation parameters (FIG. 7A). In particular, the most improved results were compositions with a PEG-lipid composition of less than 0.05 molar ratio. The size was not significantly correlated to other components of the formulation in terms of molar amounts. Similar findings have been reported where PEGylated liposomes showed a significant decrease in size compared to conventional liposomes, and that the increasing the overall amount of DSPE-PEG led to a decrease in liposome size (Kontogiannopoulos et al., 2014; Sriwongsitanont and Ueno, 2004). A potential explanation for this finding could be due to the fact that lateral repulsion of the surface of lipid bilayers increases by extensive hydration around the head group with an increasing concentration of PEG-lipid (Akinc et al., 2009). To reduce the high lateral repulsion, particle sizes must decrease, which subsequently increases the curvature of the grafting surface (Sriwongsitanont et al., 2004). In contrast, as shown here in post-nebulization formulations, a statistically significant increase in particle size was observed with increasing molar amounts of PEG-lipid (FIG. 7C). This is likely due to the type of PEG-lipid used in the formulation rather than the PEG-lipid molar ratio. From the data in FIG. 7C rearranged by type of PEG-lipid (FIG. 7D), formulations with DSPE-PEG showed a larger size compared to formulations with the other two types of PEG-lipid, which indicated that the type of PEG-lipid significantly affected the size of LNP after nebulization (FIG. 7D). In particular, in compositions without calcium, DSPE-PEG appeared lead to reduced stability during nebulization These results indicated that formulations made with DSPE-PEG had a poor ability to maintain their size after the aerosolization process.

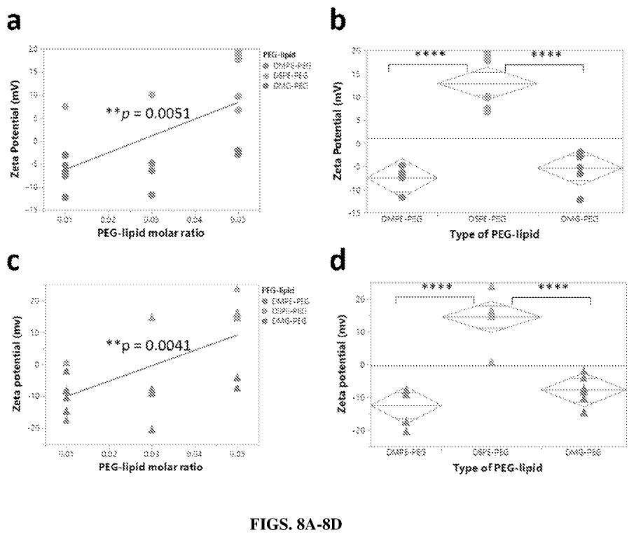

The zeta potential of the formulations, both before and after nebulization, was also primarily driven by the type of PEG-lipid selected. A statistically significant trend of increasing LNP zeta potential was observed with an increasing molar ratio of PEG-lipid for either pre-nebulized or nebulized LNP formulations, independent of the other formulation parameters (FIGS. 8A & 8C). However, it is worth noting that this significant trend was primarily related to the type of PEG-lipid used, where formulations with DSPE-PEG showed a higher zeta potential irrespective of aerosolization process (FIGS. 8B & 8D).

With respect to encapsulation efficiencies, almost all the formulations achieved high encapsulation efficiency. It was found that an increasing cholesterol molar ratio resulted in a statistically significant increase in the encapsulation efficiency for the pre-nebulized LNPs (FIG. 9A). In particular, compositions with greater than 0.1 and less than 0.49 molar ratio of the composition of cholesterol appeared to show the most promise. Furthermore, ionizable lipids in the range of 0.4 to 0.6 molar ratio showed improved properties. This indicated that the structural cholesterol played an important role in the encapsulation efficiency of LNP formulations before aerosolization, while the type of phospholipid used did not demonstrate significant effects before nebulization (FIG. 9B). Li et al. have reported that lipid-like nanoparticles with higher molar ratios of cholesterol possessed a higher encapsulation efficiency of mRNA (Li et al., 2015). However, after nebulization, the type of phospholipid, instead of the molar amount of cholesterol, became the only factor that significantly influenced the encapsulation efficiency (FIGS. 9C & 9D). LNP formulations with DOPE showed a significantly higher encapsulation efficiency compared to LNP formulations with either DSPC or DPPC (FIG. 9D). Additionally, the amount of the phospholipid may be greater than 0.1 molar ratio of the composition. This finding indicated that the inclusion of DOPE could significantly enhance the ability of LNPs to prevent mRNA from leaking during the aerosolization process.

PEG-lipid molar ratio negatively influenced the intracellular uptake of LNPs before and after nebulization. Formulations of the mRNA loaded LNPs must balance several performance measures, such as transfection efficiencies and nanoparticle stability. In the formulations developed in this study, PEG-lipids were used to impart physical stability on the nanoparticle dispersion. However, it has been shown that PEGylation can significantly influence transfection efficiencies (Otsuka et al., 2003; Mishra et al., 2004; Osman et al., 2018). Here, the PEG-lipid molar ratio significantly and negatively affected the intracellular uptake of LNPs both before and after nebulization.

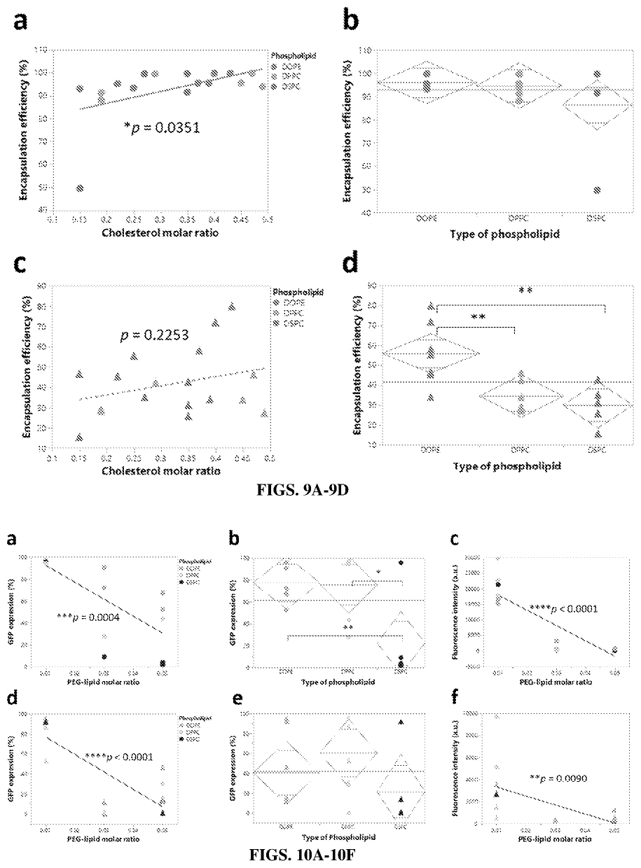

Specifically, increasing the PEG-lipid molar ratio negatively affected the intracellular uptake of pre-nebulized LNPs in HEK-293 cells (FIGS. 10A & 10C) and NuLi-1 cells (data not shown). A statistically significant trend of decreasing percent GFP expression and fluorescence intensity was observed with an increasing molar fraction of PEG-lipid, independent of the other formulation parameters; this finding was consistent with previous reports (Otsuka et al., 2003; Mishra et al., 2004). In addition, the type of phospholipid significantly influenced percent GFP expression. LNP formulations with DSPC showed significantly lower percent GFP expression compared to LNP formulations with either DOPE or DPPC (FIG. 10B), an observation consistent with previous reports (Kauffman et al., 2015). Upon nebulization, increasing the molar ratio of PEG-lipid resulted in the same observed trend in HEK-293 (FIGS. 10D & 10F) and NuLi-1 cells (data not shown), but there were no significant effects of the type of phospholipid on percent GFP expression (FIG. 10E).

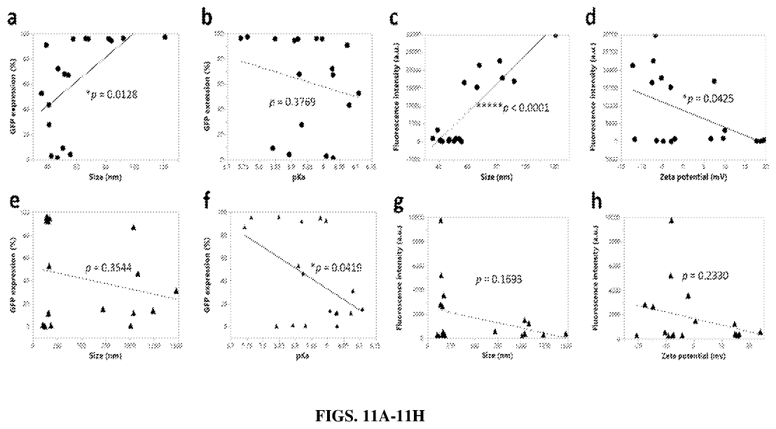

Correlation between physicochemical properties and intracellular uptake before and after nebulization. In order to explore the correlation between physicochemical properties and the potency of LNP formulations, size, zeta potential, encapsulation efficiency, and pKa were plotted against intracellular uptake and fluorescence intensity in HEK-293 cells. It was found that LNP formulations with a larger particle size showed a higher percent GFP expression and fluorescence intensity before nebulization (FIGS. 11A & 11C) as a significant trend of an increased percent GFP expression and fluorescence intensity was observed with an increased particle size (FIG. 11A). Furthermore, pre-nebulized formulations with a higher zeta potential showed a lower fluorescence intensity (FIG. 11D). After nebulization, the pKa appeared to be the significant parameter influencing percent GFP expression, whereby a lower pKa led to a higher percent GFP expression (FIG. 11F), while other parameters showed no significant effects on the intracellular uptake.

Conclusion. The in vitro performance formulations of LNPs for aerosol gene delivery are significantly influenced by lipid composition. Using the DOE approach for formulation discovery, four lead formulations that had relatively higher intracellular uptake before and after nebulization were identified and subsequently tested in vivo. These formulations when intratracheally delivered to mice, showed the ability to deliver mRNA to lungs in vivo both before and after nebulization. Extensive statistical analysis of formulations helped identify certain parameters that impacted stability and intracellular delivery of nanoparticles. DSPE-PEG was a negative factor for the stability of LNP nanoparticles as a significantly higher aggregate level appeared after nebulization compared to formulations with DMG-PEG and DMPE-PEG. It was also found that the PEG-lipid molar ratio and DSPC phospholipid significantly and negatively affected the intracellular uptake of LNPs. From this approach, LNP formulations can be more rapidly and easily identified that possess the optimal properties to facilitate effective aerosolized delivery of mRNA. While this work focused on the delivery of mRNA towards the treatment of pulmonary diseases, the DOE strategy could be broadly applied to discover LNP compositions and their properties that promote enhanced delivery of nucleic acid therapeutics for different indications.

### C. Comparative Examples

### 1. Materials

1,2-dioleoyl-3-trimethylammonium-propane (DOTAP), 1,2-dioleoyl-3-dimethylammonium-propane (DODAP), 1,2-dipalmitoyl-sn-glycero-3-phosphocholine (DPPC), 1,2-distearoyl-sn-glycero-3-phosphocholine (DSPC), 1,2-distearoyl-sn-glycero-3-phosphoethanolamine-N-[Amino (Polyethylene Glycol) 2000 (DSPE-PEG 2000), and (Delta 9 cis)/1,2-dioleoyl-sn-glycero-3-phosphoethanolamine (DOPE) were purchased from Avanti Polar Lipids, AL, USA. N-(methylpolyoxyethyleneoxycarbonyl)-1,2-dimyristoyl-sn-glycero-3-phosphoehtanolamine (DMPE-PEG 2000) was purchased from NOF Corporation, Tokyo, JP. Cholesterol was purchased from Sigma Aldrich, MO. Ethanol (molecular grade) was purchased from Decon Laboratories, Inc., PA. Edit-R Cas9 Nuclease mRNA with EGFP reporter (reference CAS11860) was purchased from Horizon Discovery Dharmacon Inc., Chicago, IL, USA. Slide-A-Lyzer™ Gamma Irradiated Dialysis Cassette (10 kDa), Quanit-iT™ RiboGreen® RNA Reagent and Kit (Invitrogen), and Opti-MEM™ Reduced Serum Media (Gibco) were purchased from ThermoFisher Scientific Inc., Waltham, MA, USA. Dulbecco's Modification of Eagle's Medium (DMEM), Fetal Bovine Serum (FBS), and Penicillin/Streptomycin (100X) were purchased from Corning, Manassas, VA, USA.

### 2. Methods

Preparation of LNP formulations. Lipid nanoparticles containing Edit-R Cas9 Nuclease mRNA were prepared by combining an aqueous phase (mRNA diluted in 50 mM sodium acetate citrate buffer, pH 4.0) and an organic phase containing ethanol and lipids according to each formulation (Table 1) using a microfluidic mixer (Precision Nanosystems, Canada; Leung et al., 2015). Flow ratio was 3:1 (aqueous:organic) and the nitrogen to phosphorus (N/P) ratio was 6. After preparation, LNP formulations were dialyzed into 1X PBS (pH 7.4) for 2 hours in 10K MWCO Slide-A-Lyzer dialysis cassettes (Thermo Fisher Scientific, MA).

Measurements of size and zeta potential. The size and zeta potential of LNP formulations were characterized by using Zetasizer Nano-ZS (Malvern Instruments MA). Each formulation was 10-fold diluted in 0.1X PBS buffer for size measurement and 40-fold diluted in 0.1X PBS for zeta potential measurement. Dynamic light scattering was performed on diluted samples at 25° C. with 173° and the reported z-average diameter is the mean of three measurements.

mRNA Encapsulation efficiency. mRNA encapsulation efficiency was evaluated by low range Quanti-iT RiboGreen RNA reagent assay (Thermo Fisher Scientific, MA). Each LNP sample was diluted into TE buffer down to a mRNA concentration of 0.2 ng/μL. Aliquots of each LNP working solution was further diluted 1:1 in TE buffer (measuring unencapsulated mRNA) or 1:1 in TE buffer with 4% Triton-X100 (measuring total mRNA-both encapsulated within LNPs and unencapsulated free mRNA) in a 96-well plate. Samples were prepared in duplicate and 100 μl of 2000-fold diluted Quanti-iT™ RiboGreen RNA reagent was added to each sample the fluorescence intensity was measured by plate reader at excitation and emission wavelengths of 480 and 520 nm (Infinite M200, Tecan, Switzerland), respectively.

Cell culture. HEK-293 cells were cultured with Dulbecco's Modified Eagle Medium containing 10% FBS and 1% penicillin streptomycin. NuLi-1 cells (ATCC CRL-4013) were cultured in flasks pre-coated with 60 μg/mL solution of human placental collagen type IV (Sigma Aldrich, MO) and grown in bronchial epithelial growth medium (BEGM) supplemented with SingleQuot additives from Lonza (BEGM Bullet Kit, reference CC-3170) and 50 μg/mL G-418. All cell lines were maintained as monolayer cultures at 37° C. and 5% CO₂.

Intracellular uptake in vitro. Cells were seeded in 96-well plates at a cell density of 12,500 cells/well and grown for 24 hours at 37° C. and 5% CO₂. Then 10 μL of LNP at a 10 ng EGFP mRNA/uL concentration was added to cells in 0.2 mL cell culture media for 24 hours. After, the cell culture media was removed, and cells were washed with 1X PBS. To detach the cells, 100 μL of 0.25% trypsin-EDTA solution was added to each well and incubated at 37° C. for 8-10 minutes. Next, 100 μL of 1% FBS in Dulbecco's phosphate buffered saline was added, cells were spun at 125 x g for 5 to 10 minutes and the supernatant was discarded. Cells were resuspended in 50 μL 1X PBS with 0.25 μL propidium iodide (PI) (1 mg/mL) solution. Cell percent GFP expression (i.e. transfection efficiency) and fluorescence intensity were analyzed by flow cytometry.

### 3. Results

Based on a mixture design with constraints, 20 formulations with an N/P ratio=6 were prepared using the NanoAssemblr® benchtop system (Table 4).

TABLE 4
Composition of LNP formulations.

| | | | | Molar composition | | | |
|---|---|---|---|---|---|---|---|
| Formulation | Cationic | | | Cationic | | | |
| # | lipid | Phospholipid | PEG-lipid | lipid | Phospholipid | PEG-lipid | Cholesterol |
| 1 | DOTAP | DPPC | DSPE-PEG | 0.44 | 0.2 | 0.05 | 0.31 |
| 2 | DODAP | DPPC | DMPE- | 0.5 | 0.2 | 0.01 | 0.29 |
| | | | PEG | | | | |
| 3 | DODAP | DOPE | DSPE-PEG | 0.4 | 0.2 | 0.01 | 0.39 |
| 4 | DOTAP | DOPE | DMPE- | 0.4 | 0.2 | 0.01 | 0.39 |
| | | | PEG | | | | |
| 5 | DODAP | DPPC | DSPE-PEG | 0.6 | 0.2 | 0.05 | 0.15 |
| 6 | DODAP | DOPE | DSPE-PEG | 0.5 | 0.1 | 0.05 | 0.35 |
| 7 | DODAP | DOPE | DMPE- | 0.6 | 0.2 | 0.01 | 0.19 |
| | | | PEG | | | | |
| 8 | DODAP | DOPE | DMPE- | 0.5 | 0.1 | 0.01 | 0.39 |
| | | | PEG | | | | |
| 9 | DOTAP | DOPE | DSPE-PEG | 0.6 | 0.2 | 0.05 | 0.15 |
| 10 | DOTAP | DOPE | DSPE-PEG | 0.6 | 0.1 | 0.01 | 0.29 |
| 11 | DOTAP | DPPC | DMPE- | 0.6 | 0.1 | 0.01 | 0.29 |
| | | | PEG | | | | |
| 12 | DODAP | DOPE | DMPE- | 0.4 | 0.2 | 0.05 | 0.35 |
| | | | PEG | | | | |
| 13 | DOTAP | DPPC | DMPE- | 0.6 | 0.2 | 0.05 | 0.15 |
| | | | PEG | | | | |
| 14 | DODAP | DPPC | DMPE- | 0.4 | 0.1 | 0.05 | 0.45 |
| | | | PEG | | | | |
| 15 | DOTAP | DPPC | DSPE-PEG | 0.6 | 0.2 | 0.01 | 0.19 |
| 16 | DODAP | DPPC | DSPE-PEG | 0.4 | 0.1 | 0.01 | 0.49 |
| 17 | DOTAP | DPPC | DSPE-PEG | 0.4 | 0.1 | 0.01 | 0.49 |
| 18 | DOTAP | DOPE | DMPE- | 0.4 | 0.1 | 0.05 | 0.45 |
| | | | PEG | | | | |
| 19 | DODAP | DOPE | DMPE- | 0.6 | 0.1 | 0.05 | 0.25 |
| | | | PEG | | | | |
| 20 | DOTAP | DPPC | DSPE-PEG | 0.6 | 0.1 | 0.05 | 0.25 |

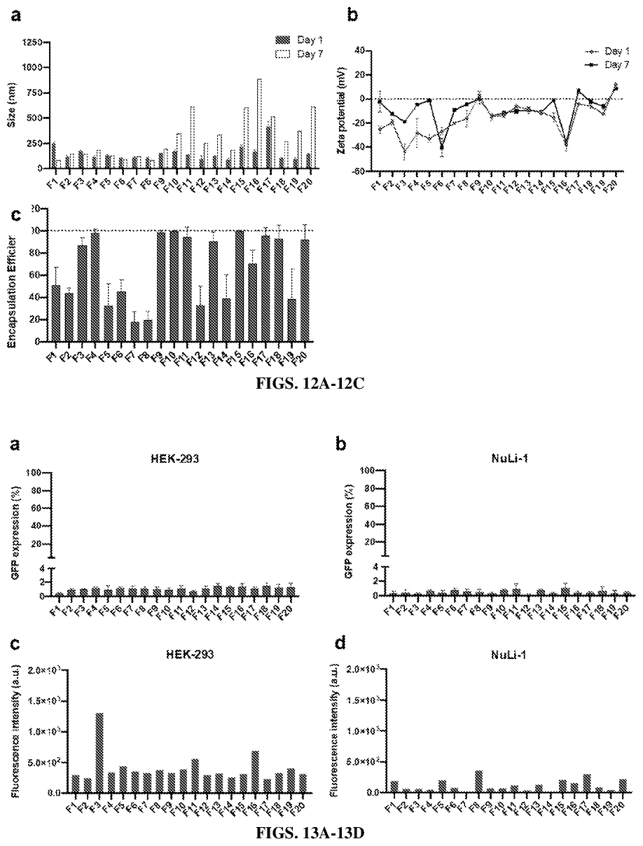

Characterization of mRNA-LNPs. The size and zeta potential of the LNPs were evaluated by dynamic light scattering (DLS) (Zetasizer Nano, Malvern Instruments, MA). Size and zeta potential measurements were performed in 0.1 X PBS at 25° C. and a scattering angle of 173°. As shown in FIG. 12A, the particle size of the LNP formulations on day 1 varied from 83.3±14.7 nm (F8) to 416.30±41.1 nm (F17), while the zeta potential ranged from −43.95±4.75 mV (F3) to 11.7±1.4 mV (F20) (FIG. 12B). However, the size and zeta potential of the LNP formulations showed changes after 7 days of storage at 4° C., with an increase in particle size and changes in zeta potential for some formulations (FIGS. 12A & 12B). The encapsulation efficiency of the formulations was evaluated by RiboGreen assay according to the manufacturer protocol (Thermo Fisher Scientific, MA). Half of the formulations possessed a high encapsulation efficiency greater than 80% (F3, F4, F9, F10, F11, F13, F15, F17, F18, and F20), and F16 demonstrated an encapsulation efficiency of 70.28%. However, the other formulations demonstrated encapsulation efficiencies equal or lower than 50% (FIG. 12C).

Intracellular uptake of LNP formulations in HEK-293 and NuLi-1 cells. The intracellular uptake of LNP mRNA formulations after 24 hs was assessed using flow cytometry by measuring percent GFP expression and fluorescence intensity in HEK-293 and NuLi-1 cell lines. It was found that all formulations showed less than 2% GFP expression (FIGS. 13A & 13B). Notably, although most formulations had relatively low percent GFP expression, the intracellular uptake in terms of fluorescence intensity varied among the formulations. F3 showed a significantly higher fluorescence intensity compared to F2, F14, and F17 in HEK-293 cells (FIG. 13C, p<0.05), but showed no significant differences in fluorescence intensities when tested in NuLi-1 cells (FIG. 13D).

### Example 2

### Development of Lipid Nanoparticle-mRNA Formulations for Pulmonary Delivery

### A. Material and Methods

### 1. Materials

Poly (ethylene glycol) monomethyl ether MW 5000 kDa, mannitol, sucrose, trehalose, and leucine were purchased from Sigma-Aldrich (St. Louis, MO, USA). Low molecular weight chitosan MW 15 kDa, was obtained from Polysciences Inc., USA. Nuclease-free water, Dulbecco's Modified Eagle's Medium (DMEM), Opti-MEM, and diethyl ether were obtained from Thermo Fisher Scientific Inc. (Waltham, MA, USA). pSpCas9(BB)-2A-GFP (PX458) was a gift from Feng Zhang (Addgene plasmid #48138; http://n2t.net/addgene:48138; RRID:Addgene_48138; Ran et al., 2013).

### 2. Methods

Preparation of LNP formulations. Lipid nanoparticles containing enhanced green fluorescent protein (EGFP) mRNA were prepared by combining an aqueous phase (mRNA diluted in 100 mM sodium acetate citrate buffer, pH 3.0) and an organic phase containing ethanol and lipids according to each formulation (Table 5) using a microfluidic mixer (Precision Nanosystems, Canada; Leung et al., 2015). After preparation, LNP formulations were dialyzed into 1X PBS (pH 7.4) for 2 hours in 10K MWCO Slide-A-Lyzer dialysis cassettes (Thermo Fisher Scientific, MA).

TABLE 5
Composition of LNP formulations.

| | | | Molar composition | | | |
|---|---|---|---|---|---|---|
| | | | Dlin-MC3- | | | |
| Formulation | Phospholipid | PEG-lipid | DMA | Phospholipid | PEG-lipid | Cholesterol |
| LNP-1 | DOPE | DMPE-PEG | 0.4 | 0.2 | 0.01 | 0.39 |
| LNP-2 | DPPC | DMG-PEG | 0.6 | 0.2 | 0.01 | 0.19 |
| LNP-3 | DPPC | DMPE-PEG | 0.6 | 0.2 | 0.01 | 0.19 |
| LNP-4 | DOPE | DMG-PEG | 0.4 | 0.16 | 0.01 | 0.43 |

Statistical analysis. The statistical analysis was performed using JMP 13. All experiments were performed in triplicate. Data values are expressed as mean±standard deviations (SD). When required, Student's t-test or one-way analysis of variance (ANOVA) was performed. *p-values<0.05 were considered statistically significant.

### Example 3

### Spray dried Lipid Nanoparticle-mRNA Formulations for Pulmonary Delivery

Lipid nanoparticles loaded with therapeutic nucleic acids obtained from examples 1 and 2 can be further processed by spray drying. Lipid nanoparticles containing mRNA were prepared by combining an aqueous phase (mRNA diluted in 100 mM sodium acetate citrate buffer, pH 3.0) and an organic phase containing ethanol and lipids according to each formulation (see for example Tables 4 and 5) using a microfluidic mixer (Precision Nanosystems, Canada; Leung et al., 2015). After preparation, LNP formulations were dialyzed into 1X PBS (pH 7.4) for 2 hours in 10K MWCO Slide-A-Lyzer dialysis cassettes (Thermo Fisher Scientific, MA). Following dialysis formulations were then spray dried.

Atomization of LNPs was performed using a two-fluid pneumatic nozzle with 0.7 mm nozzle and 1.5 mm nozzle cap (BUCHI). The cleaning needle was removed from the nozzle to prevent the generation of negative pressure from disrupting the flow rate. Compressed nitrogen was used as the atomization gas, and the atomization air flow rate was set using a flow meter positioned at the nozzle exit (Copley Scientific). The feed flow rate was set using a syringe pump (KD Scientific). Spray drying was performed with a BUCHI B-290 spray dryer with de-humidifier attachment. Aspiration rate was set at 100% and inlet temperature was set at 130° C.

### Example 4

### Freeze Drying Followed by Jet Milling of Lipid Nanoparticle-mRNA Formulations for Pulmonary Delivery

Lipid nanoparticles loaded with therapeutic nucleic acids obtained from examples 1 and 2 can be further processed by freeze drying. Lipid nanoparticles containing mRNA were prepared by combining an aqueous phase (mRNA diluted in 100 mM sodium acetate citrate buffer, pH 3.0) and an organic phase containing ethanol and lipids according to each formulation (see for example Tables 4 and 5) using a microfluidic mixer (Precision Nanosystems, Canada; Leung et al., 2015). After preparation, LNP formulations were dialyzed into 1X PBS (pH 7.4) for 2 hours in 10K MWCO Slide-A-Lyzer dialysis cassettes (Thermo

Fisher Scientific, MA). Following dialysis formulations were then. Once frozen, the liquid nitrogen formulation slurry was poured into glass vials, loosely covered and loaded into a pre-chilled VirTis BenchTop shelf lyophilizer (SP Scientific) and lyophilization was performed according to a previously published method (Sahaijipijarn et al., 2019). After the secondary drying step, the lyophilized chamber was filled with nitrogen gas and the vials were completely closed with rubber stoppers using compressed air inside the chamber. The vials were then sealed with aluminum caps. Powder was then milled using a jet mill using methods as described in 1. Implementation of design of experiments approach for the micronization of a drug with a high brittle-ductile transition particle diameter. (Yazdi and Smyth, 2016; Yazdi and Smyth 2017).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Abdelwahed et al., Adv. Drug Deliv. Rev., 58 (15): 1688-1713, 2006.
Akinc et al., Mol. Ther. 17:872-879, 2009.
Aldayel et al., J. Control. Release, 283:280-289, 2018.
Belliveau et al., Mol. Ther. Nucleic Acids, 1:e37, 2012.
Chung et al., Int. J. Pharm., 437 (1-2): 42-50, 2012.
D′Addio et al., Pharm. Res., 30 (11): 2891-2901, 2013.
Desai and Vadgama, Analyst, 116 (11): 1113, 1991.
Deshpande et al., AAPS PharmSci., 4 (3): 12-21, 2002.
Elhissi et al., Int. J. Pharm., 444:193-199, 2013.
Engstrom et al., Pharm. Res., 25 (6): 1334-1346, 2008.
Ferrati et al., AAPS PharmSciTech., 27:1-2, 2018.
Huckaby and Lai, Adv. Drug Deliv. Rev., 124:125-139, 2018.
Hyde et al., Hum. Gene Ther. Clin. Dev., 25 (2): 97-107, 2014.
Jayaraman et al., Angew. Chem. Int. Ed., 51:8529-8533, 2012.
Kauffman et al., Nano Lett., 15:7300-7306, 2015.
Kontogiannopoulos et al., J. Liposome Res., 24:230-240, 2014.
Lball et al., Int. J. Nanomed., 12:305-315, 2017.
Leal et al., Int. J. Pharm., 553 (1-2): 57-64, 2018.
Leung et al., J. Phys. Chem. B, 119:8698-8706, 2015.
Li et al., Nano Lett., 15:8099-8107, 2015.
Lintingre et al., Soft Matter, 12 (36): 7435-7444, 2016.
Mishra et al., Eur. J. Cell Biol., 83:97-111, 2004.
Nemati et al., AAPS PharmSciTech, 20 (3): 1-9, 2019.
Niu and Panyam, J. Control. Release, 248, 125-132, 2017.
Norris and Sinko, J. Appl. Polym. Sci., 63 (11): 1481-1492, 1997.
Ohashi et al., J. Control. Release, 135 (1): 19-24, 2009.
Osman et al., J. Controlled Rel., 285:35-45, 2018.
Otsuka et al., Adv. Drug Deliv. Rev., 55:403-419, 2003.
Overhoff et al., Eur. J. Pharm. Biopharm., 65 (1): 57-67, 2007.
Overhoff et al., Pharm. Res., 25 (1): 167-175, 2008.
Patil-Gadhe and Pokharkar, Int. J. Pharm., 501 (1-2): 199-210, 2016.
Patlolla et al., J. Control. Release, 144 (2): 233-241, 2010.
Rahimpour and Hamishehkar, Adv. Pharm. Bull., 2 (2): 183-187, 2012.
Ran et al., Nat. Protoc., 8 (11): 2281-2308, 2013.
Sahakijpijarn et al., Pharmaceutics 11 (10), 2019.
Sriwongsitanont and Ueno, Colloid Polymer Sci., 282:753-760, 2004.
Thakkar et al., Hum. Vaccines Immunother., 13 (4): 936-946, 2017.
Wagner et al., J. Liposome Res., 16:113-125, 2006.
Wang et al., J. Microencapsul., 35 (3): 241-248, 2018.
Yazdi and Smyth, Drug Development and Industrial Pharmacy 43 (3), 2017
Yazdi and Smyth, International Journal of Pharmaceutics, 502 (1-2): 170-180, 2016
Zhang et al., Eur. J. Pharm. Biopharm., 82 (3): 534-544, 2012.
Zhang et al., Mol. Pharm., 15 (11): 4814-4826, 2018.
Zhu et al., Mol. Pharm., 10 (9): 3525-3530, 2013.

1. A composition comprising a biologically active polynucleotide molecules and a lipid nanoparticles (LNPs), wherein the LNPs comprises at least an ionizable lipid, at least a first phospholipid and at least a first PEG-lipid, wherein the composition is formulated for inhalation.

2. (canceled)

3. The composition of claim 1, wherein the biologically active polynucleotide molecules comprises RNA.

4. The composition of claim 1, wherein the biologically active polynucleotide molecules comprises a mRNA.

5-9. (canceled)

10. The composition of claim 1, wherein the biologically active polynucleotide molecules are encapsulated in the LNPs.

11-14. (canceled)

15. The composition of claim 1, wherein the LNPs comprise a molar ratio of ionizable lipid of from about 0.4 to about 0.6; PEG-lipid of from about 0.01 to about 0.05; phospholipid of from about 0.1 to about 0.2 and cholesterol of from about 0.15 to about 0.49.

16-19. (canceled)

20. The composition of claim 1, further comprising a pH buffering agent.

21. The composition of claim 1, wherein the LNPs comprise ionizable lipids, phospholipids, cholesterol, lecithin and/or poly-(ethylene) glycol (PEG)-lipid.

22-23. (canceled)

24. The composition of claim 1, wherein the composition further comprises at least a first excipient.

25. The composition of claim 24, wherein the first excipient comprises a sugar or sugar alcohol.

26-27. (canceled)

28. A nebulized composition in accordance with the composition of claim 1.

29. A dry powder composition of claim 1, said dry powder comprising at least a first excipient, said dry powder having been produced by spray drying, spray freeze drying, or freeze drying.

30-32. (canceled)

33. The dry powder of claim 29, wherein the powder has a surface area of about 2.0 to 8.5 m²/g.

34. The dry powder of claim 29, wherein the first excipient comprises a sugar, or sugar alcohol.

35. (canceled)

36. The dry powder of claim 29, wherein first excipient comprises lactose, trehalose, sucrose, mannitol or sorbitol.

37. (canceled)

38. The dry powder of claim 29, wherein the first excipient comprises about 50%-99.5% of the powder by weight.

39-40. (canceled)

41. The dry powder of claim 29, further comprising at least a second, third and/or fourth excipient.

42-46. (canceled)

47. An inhaler comprising the composition of claim 1.

48. The inhaler of claim 47, wherein the inhaler is a fixed dose combination inhaler, a single dose dry powder inhaler, a multi-dose dry powder inhaler, multi-unit dose dry powder inhaler, a metered dose inhaler, or a pressurized metered dose inhaler.

49-53. (canceled)

54. A method treating a lung disease, lung injury or lung infection comprising administering an effective amount of a composition of claim 1 to a subject.

55-59. (canceled)
